(19) United States
(12) Patent Application Publication
(10) Pub. No.: US 2022/0168138 A1
(43) Pub. Date: Jun. 2, 2022

(54) Liquid Aloe and Polyacrylamide Cold Pack

(71) Applicants: William E. Branum, Laguna Niguel, CA (US); Richard M. Green, Hemet, CA (US)

(72) Inventors: William E. Branum, Laguna Niguel, CA (US); Richard M. Green, Hemet, CA (US)

(21) Appl. No.: 17/670,418

(22) Filed: Feb. 11, 2022

Related U.S. Application Data
(63) Continuation-in-part of application No. 14/943,298, filed on Nov. 17, 2015, now abandoned.
(60) Provisional application No. 62/127,358, filed on Mar. 3, 2015.

(51) Int. Cl. A61F 7/02; B65B 3/18; B65B 3/00

(52) U.S. Cl. CPC A61F 7/0241; B65B 3/18; A61F 2007/022; A61F 2007/0215; A61F 2007/0203; B65B 3/003

(57) ABSTRACT

Improvements in the cold pack with a sustained temperature of between 17-40 degrees Fahrenheit for over 2 hours and as long as 9 hours. The aloe and polyacrylamide cold pack can be configured so it does not continually condense or drip water for over 2 hours when using the ingredients of Ammonium C12-C15, Pareth Sulfate Ammonium C12-C15, Pareth Sulfate, Sodium Chloride, Poloxamer 124 DMDM Hydantoin Pent sodium Pentatate, Sodium Bisulfite. The main ingredients in regulating the ice pack temperature, longevity and hardness is the aloe, H2O and polyacrylamide ratio.

### CROSS REFERENCE TO RELATED APPLICATION

This application is a continuation-in-part of applicant's co-pending application Ser. No. 14/943,298 filed on Nov. 17, 2015, which claims priority to provisional application Ser. No. 62/127,358 filed Mar. 3, 2015, the entire contents of which is hereby expressly incorporated by reference herein.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

This invention relates to improvements in a cold pack. More particularly, the present cold pack is Aloe in a liquid form, H2o, polyacrylamide mix that can maintain a certain temperature for an extended period. By varying the ingredients ratio and adding other materials, there can be an increase or decrease the temperature being holds. In some cases, the ration can extend the length of time the cold pack holds the desired temperature.

### Description of Related Art Including Information Disclosed Under 37 CFR 1.97 and 1.98

For athletes and people who require cold therapy to relieve pain or promote healing, a cold pack that can be placed directly onto the surface of the skin and provides relief. Cold packs have taken a variety of types. Early types of cold packs simply use ice cubes placed in a bag or rag. More modern cold packs utilize chemicals to allow the cold pack to be more flexible and extend the period the pack remains useful.

Several patents and or publications have been made to address these issues. Exemplary examples of patents and or publication that try to address this/these problem(s) are identified and discussed below.

U.S. Pat. No. 5,478,988 issued on Dec. 26, 1995 to Thomas E. Hughes et al, discloses a Thermal Exchange Composition and Articles for use thereof. This cold pack uses a thermal exchange composition made from clay, glycol and/or water for retaining and slowly releasing hot and cold temperature. The formulation provides short term stability, not long-term stability of 4 or more hours with changes in the ratios and additives and as always condensation forms when in use.

U.S. Pat. Nos. 5,723,063 and 6,051,159 issued on Mar. 3, 1998 and Apr. 18, 2000, respectively, both to Jie Hao disclose Soft Ice. These patents are a composition of matter comprising 70% to 80% water, 5-10% salt, 12-15% glycerine and 3-5% polyacrylamide in an enclosed flexible bag. While the soft ice provides a flexible cooling therapy device, the chemical composition provides limited cold thermal longevity.

U.S. Pat. No. 6,610,084 issued on Aug. 26, 2003, to Ernesto A. Torres discloses a Shapeable Pack for Cold Therapy. The pack comprises a plurality of encapsulated units, wherein a mixture of a NaCl based salt dissolved in water that is encapsulated in polyacrylamide.

What is needed is a long lasting (over 3 hours) ice pack made of liquid aloe (used for temperature stability), polyacrylamide (for conforming fit), and H2O. The aloe and polyacrylamide water cold pack in this document provides the solution.

### BRIEF SUMMARY OF THE INVENTION

It is an object of the liquid aloe and polyacrylamide cold pack to be lightweight. It covers a large area while being 40% lighter than other products of similar size. Because the aloe and polyacrylamide cold pack is light weight, a larger surface area of the body can be covered by the product without adding too much pressure to the area being treated. The formula is used it has industrial applications in transporting perishable items.

It is an object of the liquid aloe and polyacrylamide cold pack to provide a cold pack that does not cause freezer burn in direct contact with the exterior packaging. It does not burn the skin even in direct contact with the exterior packaging when using the therapeutic formula. Physicians recommend using cold therapy for 20 min at a sustained temperature of 40 degrees Fahrenheit. Currently this is done by a machine. Otherwise, the products are heavy, non-conforming and do not meet the temperature recommendations. The aloe and polyacrylamide cold pack is comfortable within 5 min of use. The wearer will not think that he is wearing a cold pack because the body readily tolerates the 40-degree temperature stability the product provides. The cold pack maintains a sustained temperature of between 17-40 degrees Fahrenheit for over 2 hours thereby making it very comfortable compared to freezing temperatures in some products that are stiff, heavy and numbingly painful. The product is 17 to 40 degrees Fahrenheit of cold therapy minutes out of the freezer. Currently this is done by a machine. Otherwise, you other products that are heavy, non-conforming and do not meet the temperature recommendations.

It is an object of the liquid aloe and polyacrylamide cold pack to provide a cold pack that reduces condensation. This eliminates the need for heavy absorbent materials when applying the ice pack to the body. It can be used in sensitive areas of the body where condensed water formation would be an issue to wound care. It can be used and has been used in back pain management in bed without the residue of water, which is an undesirable factor when a person falls asleep while using the cold pack. The cold pack allows the user to reduce moisture volume in the area of use.

It is another object of the liquid aloe and polyacrylamide cold pack to be conforming to the body where it can be used in medical applications, surgery, post-op, genital and anal surgery where conforming materials are preferred. Eye surgery, where cold packs in a lightweight and conforming product are mistaken as ice cubes in a towel are replaced by a product in a latex cover that conforms to the area. Since the material is very conforming knee and elbow, foot, arm, hand and any other parts of the body that cold therapy can be covered.

It is another object of the liquid aloe and polyacrylamide cold pack to be used for workers who work in high heat areas where a lightweight cooled vest could increase the safety and comfort of the worker. This can also be used to cool football players and other professionals, maintaining a cool but not cold climate in various forms like boots, gloves, modified towels etc. The cold pack controls overheating during exercise by placing the product into clothing that can be worn. Also, various occupations that are in close proximity to high heat.

It is still another object of the liquid aloe and polyacrylamide cold pack to be placed in products, the list is endless. For example, cold drink cups that don't continuously condense and drip water on you while keeping the drink cold for hours.

It is still another object of the liquid aloe and polyacrylamide pack to be used in the heat therapeutic form where it can be heated in a microwave.

Various objects, features, aspects, and advantages of the present invention will become more apparent from the following detailed description of preferred embodiments of the invention, along with the accompanying drawings in which like numerals represent like components.

### BRIEF DESCRIPTION OF THE SEVERAL VIEWS OF THE DRAWING(S)

FIG. 1 shows a chart of ice packs for commercial applications with the cold pack frozen at the start of test. Some ratios of this embodiment may or may not suppress condensation.

FIG. 2 shows temperature stability using different ratios.

FIG. 3 shows flow chart of the manufacturing process for making the liquid aloe and polyacrylamide cold pack.

FIG. 4 shows a front view of a preferred embodiment of a bag with the liquid aloe and polyacrylamide.

FIG. 5 shows a side view of a preferred embodiment of a bag with the liquid aloe and polyacrylamide.

### DETAILED DESCRIPTION OF THE INVENTION

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention but is merely representative of various embodiments of the invention. The illustrated embodiments of the invention will be best understood by reference to the drawings, wherein like parts are designated by like numerals throughout.

Item Numbers and Description

| | |
|---|---|
| 20 bag | 21 width |
| 22 height | 23 thickness |
| 30 edge(s) | 31 operable side |
| 40 vent body | 41 vent slit |
| 100 process | 101 water |
| 102 polyacrylamide | 103 wait |
| 104 mixing | 105 vent |
| 106 refrigerate | 107 apply bag |
| 108 seal bag | 109 vent bag |
| 110 aloe | 111 Ammonium C12-C15 |
| 112 sodium chloride | 113 poloxamer |
| 114 DMDM | 115 Sodium bisulfite |
| 116 dyes | |

The liquid aloe and polyacrylamide cold pack uses a combination of 28 g+/−7 g polyacrlamide, H2O 236 ml+/−30 ml and 28 ml of 100% pure+/−10% liquid aloe, or extract of aloe, (Ammonium C12-C15 1-2%+1%-2% and or Pareth Sulfate 1-2%+/−1%, Ammonium C12-C15 1%+1%−1%, Sodium Chloride 2%+1/−2%, Poloxamer 124 DMDM 1%+0-1%, Hydantoin Pent sodium Pentatate 1%+/−1%, Sodium Bisulfite 1%+/−1%, with (not necessary for appearance only) Liquitint dyes Methylchloroisothiazolinone Methylisothiazolinone less than 1%+/−1%).

The formula can maintain a temperature of 17 to 40 degrees Fahrenheit for over two hours that is conforming to the body when packaged properly. The formula does not condense appreciably until over two hours have passed and then produces only slight condensation that was not saturate a two-ply tissue. The polyacrylamide cold pack is comfortable within 5 min of use after removal from a freezer. The polyacrylamide cold pack can be lightweight with variations to the formula.

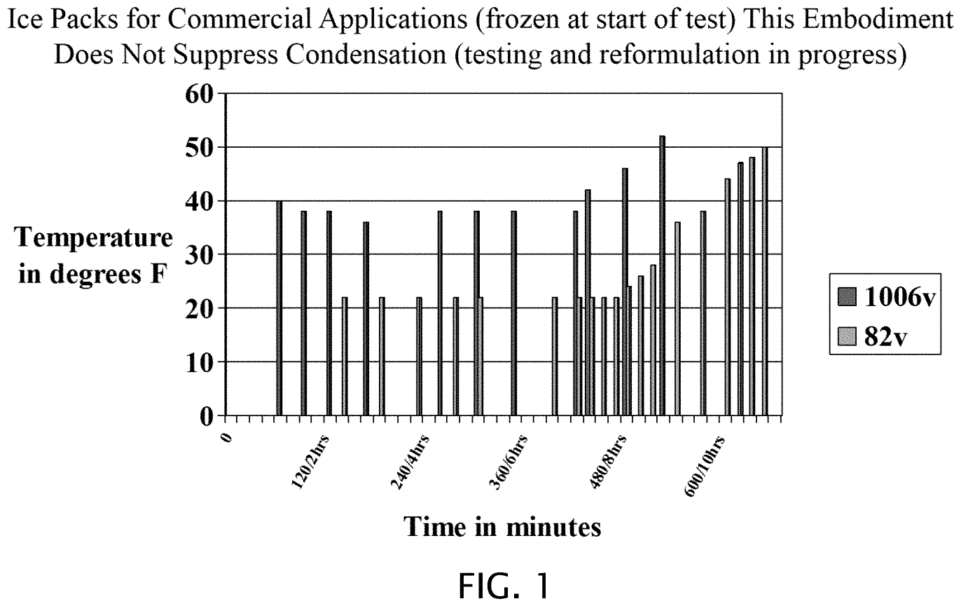

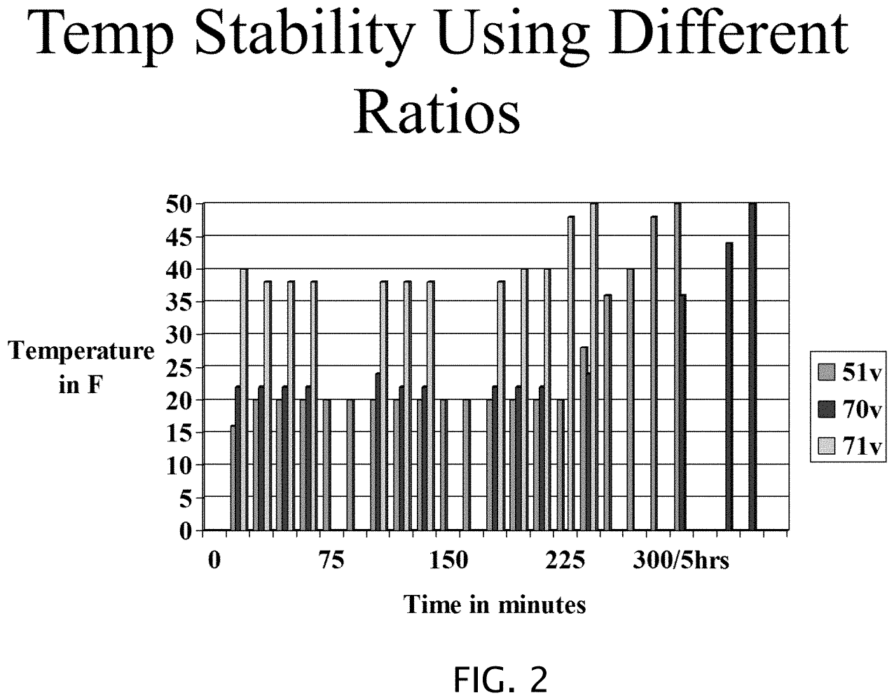

FIG. 1 shows a chart of ice packs for commercial applications with the cold pack frozen at the start of test. Some ratios of this embodiment may or may not suppress condensation and FIG. 2 shows temperature stability using different ratios. The testing is done in open air and having an ambient air temperature of 78-degree Fahrenheit temperature. In these charts, one formula is about 30 degrees Fahrenheit, and a second formula is about 40 degrees Fahrenheit. The second formula is the preferred temperature for use as a medical cold therapy produce. It is also noted that various formulations produce some temperature of less than 17 degrees and are designed for longevity and not condensation control. Thus, not requiring the additives Ammonium C12-C15, Pereth Sulfate Ammonium C12-C15, Pareth Sulfate, Sodium Chloride, Poloxamer 124 DMDM Hydantion Pent sodium Pentatate, Sodium Bisulfate.

The general formula will hold the initial temperature consistently during that time. After 2-4 hours the liquid aloe and polyacrylamide cold pack will start to breakdown and return to room temperature. It will then begin to sweat or condense while other products begin to condense almost as soon as they are removed from the freezer. In the liquid aloe and polyacrylamide cold pack the condensation is very light and will not create a high volume of moisture even in this state.

General Process Information:

Cold packs are primarily made of polyacrlamide, water and aloe. But can be made by combining the 28 g+/−7 g polyacrlamide, H2O 236 ml+/−30 ml and 28 ml of 100% pure+/−10% liquid aloe, or extract of aloe, (Ammonium C12-C15 1-2%+1%-2% and or Pareth Sulfate 1-2%+/−1%, Ammonium C12-C15 1%+1%-1%, Sodium Chloride 2%+1/−2%, Poloxamer 124 DMDM 1%+0-1%, Hydantoin Pent sodium Pentatate 1%+/−1%, Sodium Bisulfite 1%+/−1%, with (not necessary for appearance only Liquitint dyes Methylchloroisothiazolinone Methylisothiazolinone less than 1%+/−1%). The combination of ingredients are then well mixed with minimal agitation and without air in the bag.

First Process:

1a: Polyacrlamide with H2O at room temperature (65-78 degrees F.)

1b: Wait for about 75%+0/−20% of the water to be absorbed into the polyacrlamide. The approximate time for absorption is 45 seconds+30 seconds−0. The absorption of the water into the polyacrylamide basically fills the polyacrylamide to prevent (or minimize) absorption of the remaining ingredients into the polyacrylamide.

Combine:

2a: Contents of bag with formula.

2b: Remove air and foam.

2c: Mix well with minimal agitation.

2d: Form the 15 mm×14 mm×0.2 mm plastic bag into a flat rectangle and freeze the mixture at −2 degrees F. Creates a bag 15 mm×14 mm×approximately 2-4 mm thick. Produces a stable temperature between 32-40 degrees F. for 2-4 hours at room temperature (68-78 degrees F.).

The therapeutic formula of the liquid aloe and polyacrylamide cold pack will hold the initial temperature of 40 degrees+/−7 degrees F. consistently during that time. After 2-4 hours the liquid aloe and polyacrylamide cold pack will start to breakdown and return to room temperature.

Formulations or variants in the amount of Formula used in the mixture vary the stable temperature and longevity of the liquid aloe and polyacrylamide cold pack. Tests run at room temperature show that adding 28 ml of the formula drops the stable temperature from 40 degrees F. to 29 degrees F. This obviously can be modified to suit the circumstances.

The preferred ratio of water to aloe is 4 parts H2O to 3 parts formula. This creates a hard pack of iced material that is not suitable for therapy but optimized for industrial applications. Decreasing or maintaining the H2O relative to the aloe content while other factors remain within their percentages, increases the longevity, and decreases the stable temperature from 32-40 degrees to as low as 17 degrees F.+/−5 degrees and lasts for as much as 5-10 hours (at a room temperature of between 68-78 degrees F.) before the temperature reaches 50 degrees F.

Within an insulated compartment the cold pack with increase liquid aloe content and a mix ratio of 4 H2O to 3 of liquid aloe has an initial temperature of 17-25 degrees F. and after 25+ hours after freezing are still at 32 degrees F. This change in ratio drives the stable temperature and longevity of use and is necessary for applications of food/perishable storage as opposed to the initial formula that is more limited in length of useable time and the temperature it maintains (usually 32-40 degrees as opposed to 17-27 degrees in cold storage applications). Note: All factors and time to failure are based upon a max useable temperature of 50 degrees. Factors are approximate and can vary with freezing temperatures, ambient temps and insulating factors of various materials used in the tests.

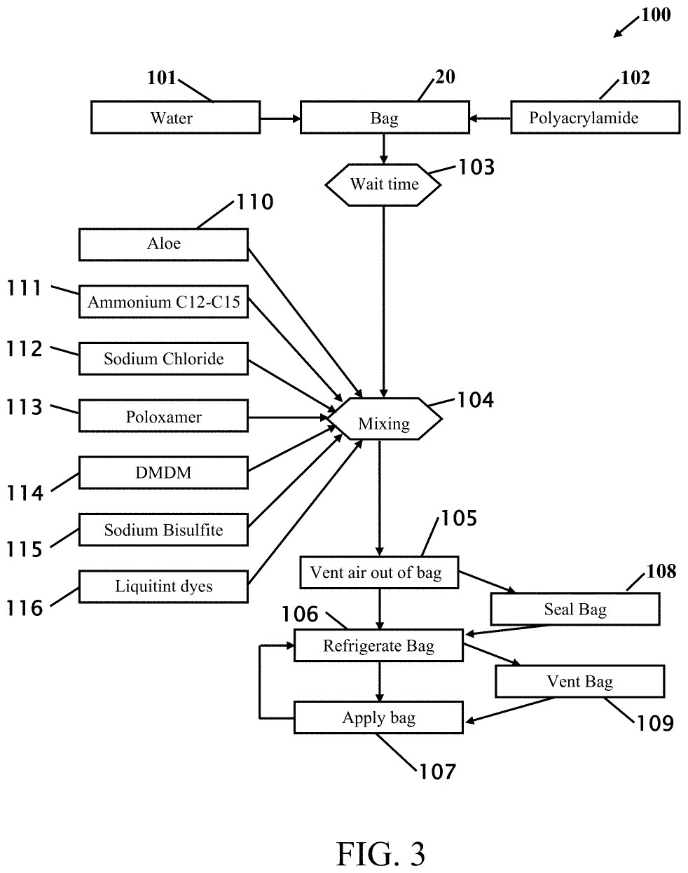

FIG. 3 shows flow chart of the manufacturing process 100 for making the liquid aloe and polyacrylamide cold pack. The process 100 begins with combining polyacrylamide 102 with water 101 at room temperature (65-78 degrees F.) into the bag 20. The combined ingredients will wait for about 75%+0/−20% of the water to be absorbed into the polyacrylamide. The approximate wait time 103 for absorption is 45 seconds+30 seconds−0. The absorption of the water into the polyacrylamide basically fills the polyacrylamide to prevent (or minimize) absorption of the remaining ingredients into the polyacrylamide.

After sufficient absorption of the water 101 into the polyacrylamide 102 the bag is combined with additional ingredients of 28 ml+/−10% of 100% pure liquid aloe 110, or extract of aloe, 0% to 3% Ammonium C12-C15 111 or Pareth Sulfate, 0%-2% Ammonium C12-C15 111, 0%-3% Sodium Chloride 112, 0%-1% Poloxamer 113, 0%-2% 124 DMDM 114 Hydantoin Pent sodium Pentatate, 0%-2% Sodium Bisulfite 115 and 0%-2% liquitint dyes 114 Methylchloroisothiazolinone Methylisothiazolinone.

The combination of ingredients are mixed 104 with squeezing, kneading or equivalent. Any air, or excess air, is then vented out of the bag 105. The bag can then be sealed 108 and is ready for use as a heating or cooling bag by refrigerating the bag 106. After refrigeration 106 (or heating) the bag can be applied 107 for therapeutic purposes or for other thermal purposes. After heating or cooling the bag can be further vented 109 to remove any air through the vent hole that is sized to prevent any expanded polyacrylamide to exit the bag, but also sized to allow air to be vented. The cooling 106 (or heating) then use can be repeated as needed/required.

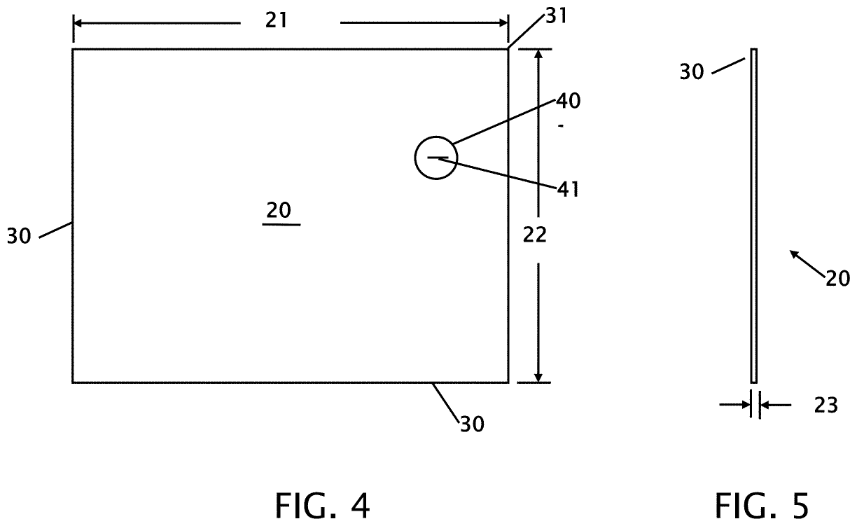

FIG. 4 shows a front view of a preferred embodiment of a bag 20 with the liquid aloe and polyacrylamide and FIG. 5 shows a side view of a preferred embodiment of a bag 20 with the liquid aloe and polyacrylamide. In one preferred embodiment the bag has flat dimensions of width 21 and height 22 of approximately 14 mm×15 mm. The thickness 23 is approximately 0.1 mm. The edges 30 of the bag 20 are sealed and the bag 20 can have an openable side 31 to mix and burp the bag 20. After the ingredients are mixed and stabilized the bag can be sealed to prevent leaking, contamination or tampering. After sealing

A re-closable bag is filled with 120 ml of water, then 15 ml of polyacrylamide is added. This mixture stands till most of the water is absorbed (approximately 75% and there is still some clear liquid is left and then add to the aloe mixed with the other ingredients of Ammonium C12-C15, Pareth Sulfate Ammonium C12-C15, Pareth Sulfate, Sodium Chloride, Poloxamer 124 DMDM Hydantoin Pent sodium Pentatate, Sodium Bisulfite, 2 tablespoons. The combination is mixed by sealing the materials inside the re-closable bag and squeezing or kneading the material till the combination is mixed and the contents are all the same color of blue. The re-closable bag is opened and air and foam is removed from the re-closable bag. The re-closable bag is sealed after removing all air and foam. Once an airtight seal is in place, the re-closable bag is formed into a flat rectangle and freezing the mixture at −2-degree Fahrenheit creates a bag 8″×7″×approximately 0.250-0.375 thick. Produces a temperature between 17-40 degree Fahrenheit for 2-4 hours at room temperature 72 degrees Fahrenheit as shown in the figure. The bag 20 is configured with a vent body 40 with a vent slit 41. The slit 41 is sized to prevent any of the expanded polyacrylamide from passing through the slit 41. The slit is sufficiently narrow to allow air from within the bag 20 to pass through the slit 41 to vent any air that remains in the sealed bag 20 as well as vent any air that exists around the polyacrylamide. The slit is configured to slightly open during freezing to allow some air to enter into the bag to allow for some air to enter in and around the freezing crystal structure.

Secondary Process Allows for a Large Lightweight Variation:

By adding water 120 ml and another 15 ml of the polyacrylamide you can increase the surface area that the material will cover and will be a lightweight material that will expand the bag to its maximum size and thickness as opposed to the initial product that is the length and the width of the bag and is approximately 0.250-0.375 in thickness. The variation increases the thickness to around 2.5 to 3.0 inches at its maximum thickness. This pack will only last 1-2 hours at temperature. Modifications to the formula may lengthen the meantime to failure of the bag for rupturing as the bag is conformed, as seen in the figures where the graph shows that we can increase the stability by hours.

Formulations or variants in the amount of aloe and crystals used in the mixture appear to vary the stable temperature. Tests run at room temperature show that adding one tablespoon of aloe mix drops the stable temperature from 40 degrees Fahrenheit to 29 degrees Fahrenheit. This obviously can be modified to suit the circumstances. Although more tests to observe the stability of the formula at the different temperatures is needed, tests already completed show the break down in both samples occur somewhere around the same 3-4-hour time frame as shown in the figures.

Thus, specific embodiments of an aloe, polyacrylamide cold pack has been disclosed. It should be apparent, however, to those skilled in the art that many more modifications besides those described are possible without departing from the inventive concepts herein. The inventive subject matter, therefore, is not to be restricted except in the spirit of the appended claims.

### SEQUENCE LISTING

Not Applicable.

1. A method of making a liquid aloe and polyacrylamide cold pack comprising:
filling a bag with 206 to 266 ml of water;
adding 21 to 35 ml of polyacrylamide to said closable bag;
allowing said polyacrylamide to absorb at least 75% of said water to become expanded polyacrylamide;
adding 18 to 38% of liquid aloe, or extract of aloe by weight;
0% to 3% Ammonium C12-C15 or Pareth Sulfate by weight;
0%-2% Ammonium C12-C15 by weight;
0%-3% Sodium Chloride by weight;
0%-1% Poloxamer by weight;
0%-2% 124 DMDM Hydantoin Pent sodium Pentatate by weight;
0%-2% Sodium Bisulfite by weight;
mixing combined ingredients in said bag until said mixed contents are essentially all the same color;
squeezing said bag to remove air from within said bag through a vent hole in said bag.

2. The method of making a liquid aloe and polyacrylamide cold pack according to claim 1, further includes sealing said bag.

3. The method of making a liquid aloe and polyacrylamide cold pack according to claim 1, wherein said mixing is with squeezing, kneading.

4. The method of making a liquid aloe and polyacrylamide cold pack according to claim 1, further includes forming said bag into a flat rectangle.

5. The method of making a liquid aloe and polyacrylamide cold pack according to claim 4, wherein said flat rectangle is configured to be between 0.125 and 0.375 inches thick.

6. The method of making a liquid aloe and polyacrylamide cold pack according to claim 4, wherein said cold pack is configured in a size of 8.000×7.000 inches and 0.250 to 0.375 inches thick.

7. The method of making a liquid aloe and polyacrylamide cold pack according to claim 1, further includes refrigerating said mixed combined ingredients in said bag.

8. The method of making a liquid aloe and polyacrylamide cold pack according to claim 1, wherein said mixed combined ingredients in said bag produces a stable temperature between 32-40 degrees F. for 2-4 hours at room temperature of between 68-78 degrees F.

9. The method of making a liquid aloe and polyacrylamide cold pack according to claim 1, wherein said absorption of said water into said polyacrylamide is performed in a time of between 45 seconds and 75 seconds.

10. The method of making a liquid aloe and polyacrylamide cold pack according to claim 1, wherein said removal of air is through a slit in said bag that is sized to allow air to pass though said slit and also sized to prevent said expanded polyacrylamide from passing through said slit.

11. The method of making a liquid aloe and polyacrylamide cold pack according to claim 1, wherein said allowing said polyacrylamide to absorb at least 75% of said water is in a time of more than 15 seconds.

12. The method of making a liquid aloe and polyacrylamide cold pack according to claim 1, wherein said allowing said polyacrylamide to absorb at least 75% of said water is in a time of less than 75 seconds

13. The method of making a liquid aloe and polyacrylamide cold pack according to claim 1, is further installed in a boot, glove, or towel.

14. The method of making a liquid aloe and polyacrylamide cold pack to claim 1, further includes installing said vent body in said bag.

15. The method of making a liquid aloe and polyacrylamide cold pack according to claim 14, further includes installing a slit in said vent body.

16. The method of making a liquid aloe and polyacrylamide cold pack according to claim 1, further include heating said liquid aloe and polyacrylamide cold pack.

17. The method of making a liquid aloe and polyacrylamide cold pack according to claim 14, wherein said slit is configured to slightly open during freezing to allow some air to enter into the bag to allow for some air to enter in and around the freezing crystal structure.

18. The method of making a liquid aloe and polyacrylamide cold pack according to claim 1, wherein said liquid aloe and polyacrylamide cold pack is configured to not continually condense, drip or lose water for said over two hours.

19. The method of making a liquid aloe and polyacrylamide cold pack according to claim 1, wherein said cold pack is configured to maintain a temperature of 17 to 40 degrees Fahrenheit for over two hours.

20. The method of making a liquid aloe and polyacrylamide cold pack according to claim 1, further includes 0%-2% liquitint dyes Methylchloroisothiazolinone Methylisothiazolinone by weight.
